(19) United States
(12) Patent Application Publication
(10) Pub. No.: US 2016/0015741 A1
(43) Pub. Date: Jan. 21, 2016

(54) Cerium Oxide Nanoparticles Accelerate the Decay of Peroxynitrite (ONOO-)

(71) Applicant: UNIVERSITY OF CENTRAL FLORIDA RESEARCH FOUNDATION, INC, Orlando, FL (US)

(72) Inventors: Sudipta Seal, Orlando, FL (US); William T. Self, Oviedo, FL (US); Janet Dowding, Winter Garden, FL (US)

(73) Assignee: UNIVERSITY OF CENTRAL FLORIDA RESEARCH FOUNDATION, INC., Orlando, FL (US)

(21) Appl. No.: 14/771,838

(22) PCT Filed: Feb. 27, 2014
(86) PCT No.: PCT/US14/18965

§ 371 Date: Sep. 1, 2015

Related U.S. Application Data
(60) Provisional application No. 61/771,137, filed on Mar. 1, 2013.
(60) Provisional application No. 61/782,649, filed on Mar. 14, 2013.

(51) Int. Cl. A61K 33/24; A61K 9/14

(52) U.S. Cl. CPC A61K 33/24; A61K 9/14

(57) ABSTRACT

Disclosed herein are methods and materials that may be implemented to scavenge a potentially destructive free radical, peroxynitrite. Specifically exemplified are cerium based nanoparticles that react with and reduce peroxynitrite. The discoveries disclosed herein reveal several therapeutic uses of such peroxynitrite reactive particles.

### CROSS-REFERENCE TO RELATED APPLICATIONS

This application is related to U.S. Provisional Patent Application No. 61/771,137 filed Mar. 1, 2013 and U.S. Provisional Patent Application No. 61/782,649 filed Mar. 14, 2013 to which priority is claimed under 35 USC 119. The teachings of these provisional applications are incorporated herein by reference in their entirety.

### STATEMENT OF FEDERAL FUNDING

The invention was made with government support under grant R01AG031529-01 awarded by the National Institutes of Health, and under NIRT CBET 0708172 awarded by the National Science Foundation. The government has certain rights in this invention.

### INTRODUCTION

Biological cells survive in a constantly changing and challenging environment, responding to many factors including oxidative byproducts of normal metabolism. Progressive accumulation of damaged molecules or tissues are believed to be responsible for the ever-increasing susceptibility to disease and death [1]. Reactive oxygen species (ROS) and reactive nitrogen species (RNS) cause damage to all types of biomolecules, including DNA, proteins, and lipids with the formation of toxic and mutagenic products. More recently, the role of RNS have been shown to have a direct role in cell signaling, vasodilation and the immune response [2]. Nitrosative stress, defined by the excessive production of reactive nitrogen species, causes damage to macromolecules and can lead to degenerative diseases, contribute to metabolic diseases and if in great excess can lead to cell death through a variety of molecular mechanisms. The role of RNS in many age related diseases, with the primary RNS species being peroxynitrite (ONOO⁻), is now just being appreciated.

CeO₂ NPs have been shown to possess a substantial oxygen storage capacity via the interchangeable surface reduction and oxidation of cerium atoms, cycling between the Ce⁴⁺ and Ce³⁺ redox states [3]. Due to this intrinsic capability, these materials have been employed for industrial use in three-way catalysts [4]. With the discovery that these nanoparticles can react effectively with biologically relevant radical species and oxidants, a new field has emerged studying these materials for use in biological systems. Biological uses of CeO₂ NPs have centered around their ability to scavenge free radicals under physiologically relevant conditions. This catalytic nature, which began with the discovery that water-based cerium oxide nanoparticles (with increased Ce³⁺ in their outer surface) could act as superoxide dismutase mimetics [3, 5], has laid the foundation for their application in experimental and biomedical research. Subsequent studies have established cerium oxide nanoparticles (with decreased Ce³⁺ in their outer surface) have the ability to act as catalase mimetics [6] as well as scavenge nitric oxide [7]. Due to their structure-function relationship to other redox catalysts (superoxide dismutase [8] and catalase [9]), Ferrer-Sueta eta/demonstrated manganese porphyrins efficiently scavenge peroxynitrite and species derived from it [10]. Since CeO₂ NPs have catalytic activity towards O₂*⁻, H₂O₂ and .NO, we hypothesized that CeO₂ NPs would be able to interact with peroxynitrite. Additionally, much of damage attributed to .NO and O₂*⁻ is actually the result of oxidation or nitration by peroxynitrite or its breakdown products [11].

### BRIEF DESCRIPTION OF THE DRAWINGS

FIG. 1 CeNP1 and CeNP2 accelerate the decay of peroxynitrite in vitro. Relative absorbance of peroxynitrite (25 μM) at 302 nm per unit time (seconds) either in the absence or presence of CeNPs nanoparticles or glutathione (GSH) (1 mM) using UV-visible spectrometry at pH 9.5. a CeNP1 (100 μM) b CeNP2 (100 μM). Relative APF (10 μM) fluorescence at 490 nm excitation and 515 nm emission wavelength with either peroxynitrite (20 μM) alone or with increasing concentrations of CeNPs nanoparticles (100, 500, 1000 μM) or glutathione (1 mM) measured over a time period of 10 seconds at pH 7.4. c CeNP1 d CeNP2. Statistics: Student's t-test, p≦0.05.

FIG. 2 CeNPs prevent 3-nitrotyrosine protein by peroxynitrite. Graphical representation of BSA western blots protected from nitration with dose dependent addition of either CeNP1, CeNP2, GSH or negative control SiO₂ NP. Inset: Representative slot-blots. All lanes contain 500 ng BSA treated with 10 μM peroxynitrite (ONOO⁻) in the absence and presence of 500 nM NPs or 1 mM GSH. Blots were probed with anti-3-nitrotyrosine antibody. Individual experiments were normalized to their individual BSA/ONOO⁻ treated lane. Data are representative of three or more independent experiments (see Online Resource—Methods and Materials). p≦0.001. Statistics: Student's t-test.

FIG. 3 Peroxide mediated changes in surface cerium oxidation state of CeNPs does not alter catalysis with peroxynitrite. Relative APF (10 μM) fluorescence at 490 nm excitation and 515 nm emission wavelength with either peroxynitrite (20 μM) alone, or in combination with CeNPs nanoparticles CeNP1 treated with H₂O₂ (100 mM) or glutathione (1 mM) measured over a time period of 10 seconds at pH 7.4. Representative traces of three or more experiments are shown (see Online Resource—Methods and Materials).

FIG. 4, Controls for peroxynitrite accelerated decay assays. Uric acid (UA) and SiO₂ NPs were added at concentrations indicated. a) Relative absorbance at 302 nm b) Relative APF fluorescence at 490 nm excitation and 515 nm emission

FIG. 5 Physical properties of Ceo2 NPs. a) Zeta potential, b) Hydrodynamic radius.

FIG. 6 CeNP1 and CeNP2 do not interfere with APF's ability to be oxidized by ONOO⁻ under inert atmosphere. Relative APF (10 μM) fluorescence at 490 nm excitation and 515 nm emission wavelength with either peroxynitrite (20 μM) alone or with 100 μM of CeNPs nanoparticles or uric acid (UA) measured after 20 min at pH 7.4. End pointAPF assay was performed under argon atmosphere. Buffers and reagents were purged by flushing with argon prior to use when possible.

### DETAILED DESCRIPTION

Embodiments described herein are based on the discovery that cerium nanoparticles can accelerate the decay of peroxynitrite, or breakdown products thereof. In one embodiment, a method of treating a subject with elevated levels of peroxynitrite, is provided, the method includes administering a therapeutically effective amount of cerium oxide nanoparticles to the subject, wherein the cerium oxide nanoparticles reduce the level of and/or accelerate the decay of, peroxynitrite, or breakdown products thereof, in the subject. In a particular embodiment, the cerium oxide nanoparticles include those that range in size between 1-20 nanometers. In a further embodiment, the cerium oxide nanoparticles from about 1 nm to about 10 nm. In another embodiment, the cerium oxide nanoparticles range between 5-10 nanometers in size. In another embodiment, the method is provided wherein the cerium oxide nanoparticles scavenge peroxynitrite.

In yet another embodiment, a method of treating a subject identified as at risk of developing a neurodegenerative disease is provided. The method includes administering a therapeutically effective amount of cerium oxide nanoparticles to the subject, wherein the cerium oxide nanoparticles accelerate decay of peroxynitrite, or breakdown products thereof.

In another embodiment, the cerium oxide nanoparticles used and which are effective to scavenge peroxynitrite are capable of accelerating the decay of peroxynitrite independent of their is independent of the Ce³⁺/Ce⁴⁺ ratio on the surface of the cerium oxide NPs. Thus, the CeO₂ NPs may have a higher ratio of 3+ to 4+ state, a lower 3+ to 4+ state, or somewherein therebetween.

As known by those having ordinary skill in the art, chemically, most of the rare earth (RE) elements (atomic numbers 57 through 71) are trivalent. Cerium alone is known to form compounds with a valence of +4, such as CeO₂ (ceria). Cerium is believed to be a unique material with regard to the mixed valence states provided, both +3 and +4. However, at least with regard to cerium oxide compounds, the vast majority of valence states are +4 states.

Cerium of valence +3 is generally referred to as cerous, while with valence +4 is generally referred to as ceric. Cerium oxide includes both ceric oxide and cerous oxide. Cerous oxide is also known as Cerium III oxide and has the formula Ce₂O₃. Ceric oxide is known as ceria, cerium dioxide and cerium IV oxide and has the chemical formula CeO₂.

The cerium oxide nanoparticles used with the invention have an average particle size <20 nm, such as in the range from 1 to 10 nanometers, for example 3 to 7 nm. The inventors have found that an average cerium oxide nanoparticle size in the range <20 nm provides an unexpected and highly beneficial result which is believed to be based on an increased percentage of +3 valence states (relative to the generally more numerous +4 states) on the cerium oxide nanoparticles surface. The increasing percentage of +3 valence states is believed to increase as the cerium oxide nanoparticle size decreases in this size range. The presence of a relatively high percentage of +3 valence states has been found to significantly improve performance of reduction of reactive nitrogen species (RNS) according to the present disclosure. A large relative percentage of +3 states in the <20 nm cerium oxide nanoparticles have also been found by the inventors to provide efficient regeneration as described below.

Neurodegenerative diseases include but are not limited to Alzheimer's disease, Parkinson's disease, Amyotrophic lateral sclerosis, Friedreich's ataxia, Huntington's disease, or Lewy body disease. A subject at risk of developing a neurodegenerative disease can be identified by detecting or observing a number of different signs and symptoms in the subject. Some of those signs and symptoms include amyloid plaques in the brain, and/or neurofibrillary tangles (NFTs) in the brain.

In the case of Alzheimer's disease, eight cognitive domains are most commonly impaired, including memory, language, perceptual skills, attention, constructive abilities, orientation, problem solving and functional abilities (70).

Also, a decrease in activity in the temporal lobe is observed in AD development, such as through the use of known imaging techniques such as PET scan or MRI. Thus, according to one embodiment, a patient at risk would be an individual who has impairment in cognition and/or decreased activity in the temporal lobe. When available as a diagnostic tool, single photon emission computed tomography (SPECT) and positron emission tomography (PET) neuroimaging are used to confirm a diagnosis of Alzheimer's in conjunction with evaluations involving mental status examination (71). In a person already having dementia, SPECT appears to be superior in differentiating Alzheimer's disease from other possible causes, compared with the usual attempts employing mental testing and medical history analysis (72).

A new technique known as PiB PET has been developed for directly and clearly imaging beta-amyloid deposits in vivo using a tracer that binds selectively to the A-beta deposits. The PiB-PET compound uses carbon-11 PET scanning. Recent studies suggest that PiB-PET is 86% accurate in predicting which people with mild cognitive impairment will develop Alzheimer's disease within two years, and 92% accurate in ruling out the likelihood of developing Alzheimer's. A similar PET scanning radiopharmaceutical compound called (E)-4-(2-(6-(2-(2-(2-([¹⁸F]-fluoroethoxy)ethoxy)ethoxy)pyridin-3-yl)vinyl)-N-methyl benzenamine, or ¹⁸F AV-45, or florbetapir-fluorine-18, or simply florbetapir, contains the longer-lasting radionuclide fluorine-18, has recently been created, and tested as a possible diagnostic tool in Alzheimer's patients. Florbetapir, like PiB, binds to beta-amyloid, but due to its use of fluorine-18 has a half-life of 110 minutes, in contrast to PiB's radioactive half life of 20 minutes. Wong et al. found that the longer life allowed the tracer to accumulate significantly more in the brains of the AD patients, particularly in the regions known to be associated with beta-amyloid deposits. Thus, in specific embodiment, a patient at risk is one that has increased a-beta deposits.

Volumetric MRI can detect changes in the size of brain regions. Measuring those regions that atrophy during the progress of Alzheimer's disease is showing promise as a diagnostic indicator. Thus, according to another specific embodiment, an at-risk patient is one that has an atrophic brain region.

Another recent objective marker of the disease is the analysis of cerebrospinal fluid for amyloid beta or tau proteins, both total tau protein and phosphorylated tau_{181P} protein concentrations. Searching for these proteins using a spinal tap can predict the onset of Alzheimer's with a sensitivity of between 94% and 100%. Thus, according to another specific embodiment, a patient at risk is one that has elevated levels of tau and/or amyloid beta proteins in cerebral spinal fluid. When used in conjunction with existing neuroimaging, doctors can identify patients with significant memory loss who are already developing the disease (73). Spinal fluid tests are commercially available, unlike the latest neuroimaging technology. Alzheimer's was diagnosed in one-third of the people who did not have any symptoms in a 2010 study, meaning that disease progression occurs well before symptoms occur. Changes in brain ventricle size may be measured by magnetic resonance imaging (MRI). This measurement provides, in another embodiment, the ability to diagnose pre-Alzheimer's disease or early stages of the disease in some cases. While neuro-cognitive assessments including the testing of memory, ability to problem solve, count, and other cognitive tests provides a diagnosis for Alzheimer's disease, a definitive diagnosis is not possible in the prior art until after death when an autopsy can be used to reveal the presence of amyloid plaques and tangles in brain tissue. Improvements have been made such that an earlier diagnosis may be made by identifying an increase in ventricle size in the brain associated with mild cognitive impairment in patients at risk for Alzheimer's disease or in the early stages of the disease. Therefore, according to a specific embodiment, a patient is at risk for a neurodegenerative disease, particularly AD, if the patient exhibits one or more of the foregoing factors or symptoms. In another specific embodiment, a patient at risk exhibits two or more of the aforementioned factors or symptoms.

In the case of Parkinson's disease (PD), a pattern of reduced dopaminergic activity in the basal ganglia can aid in diagnosis. Thus, in another specific embodiment, a patient at risk is one that has reduced dopaminergic activity in the basal ganglia. Also, Parkinson's disease affects movement, producing motor symptoms, such as Parkinsonian gait, tremors, rigidity, slowness of movement and postural instability. Non-motor symptoms, which include autonomic dysfunction, neuropsychiatric problems (mood, cognition, behavior or thought alterations), and sensory and sleep difficulties, are also common. Thus, according to another specific embodiment, a patient at risk is one that exhibits one or more motor or non-motor PD symptoms. In an even more specific embodiment, a patient at risk is one that has two or more of the foregoing factors or symptoms.

In a further embodiment, a method of reducing brain inflammation in a patient is provided. The method includes administering a therapeutically effective amount of cerium oxide nanoparticles to the patient, the cerium oxide nanoparticles effective to reduce levels of peroxynitrite, or breakdown products thereof, in the brain.

In still a further embodiment, a method of accelerating decomposition of reactive nitrogen species in a subject is provided. The method includes administering a therapeutically effective amount of cerium oxide nanoparticles to the subject, wherein the cerium oxide nanoparticles associate with a membrane in the subject and accelerate decomposition of the reactive nitrogen species in the subject. The method is provided wherein the reactive nitrogen species is peroxynitrite, or breakdown products thereof. In a more specific embodiment, the membrane is a mitochondrial membrane and/or a plasma membrane.

As used herein, the terms “subject” and “patient” are used interchangeably. As used herein, the term “subject” refers to an animal, preferably a mammal such as a non-primate (e.g., cows, pigs, horses, cats, dogs, rats etc.) and a primate (e.g., monkey and human), and most preferably a human.

As used herein, the terms “about” and “approximately” as used herein refer to values that are ±10% of the stated value.

As used herein, the terms “treating” or “treatment” or “alleviation” refers to both therapeutic treatment and prophylactic or preventative measures, wherein the object is to prevent or slow down (lessen) the targeted pathologic condition or disorder.

As used herein, the term “administering” or “administration” includes but is not limited to oral or intravenous administration by liquid, capsule, tablet, or spray. Administration may be by injection, whether intramuscular, intravenous, intraperitoneal or by any parenteral route. Parenteral administration can be by bolus injection or by continuous infusion. Formulations for injection may be presented in unit dosage form, for example, in ampoules or in multi-dose containers with an added preservative. The compositions may take the form of suspensions, solutions or emulsions in oily or aqueous vehicles and may contain formulatory agents such as suspending, stabilizing and/or dispersing agents. Alternatively the compositions may be in powder form (e.g., lyophilized) for constitution with a suitable vehicle, for example sterile pyrogen-free water, before use. Compositions may be delivered to a subject by inhalation by any presently known suitable technique including a pressurized aerosol spray, where the dosage unit may be controlled using a valve to deliver a metered amount.

Administration by capsule and cartridges containing powder mix of the composition can be used in an inhaler or insufflator to deliver the particles to the subject. Still other routes of administration which may be used include buccal, urethral, vaginal, or rectal administration, topical administration in a cream, lotion, salve, emulsion, or other fluid may also be used.

Pharmaceutical Compositions

As used herein, a “composition,” “pharmaceutical composition” “therapeutic composition” or “therapeutic agent” all include a composition comprising at least cerium oxide nanoparticles. Optionally, the “composition,” “pharmaceutical composition” “therapeutic composition” or “therapeutic agent” further comprises pharmaceutically acceptable diluents or carriers. In the case of an nanoparticles discussed herein, for example, the nanoparticles may be combined with one or more pharmaceutically acceptable diluents, such as phosphate-buffered saline, for example. As used herein, a pharmaceutical composition particularly refers to a composition comprising at least a cerium oxide nanoparticle that is intended to be administered to a subject as described herein.

The pharmaceutical composition may be prepared and administered in a wide variety of dosage formulations and may be administered orally, rectally, or by injection (e.g. intravenously, intramuscularly, intracutaneously, subcutaneously, intraduodenally, or intraperitoneally). Preparations of pharmaceutical compositions of nanoparticles may include pharmaceutical acceptable carriers that can be either solid or liquid.

Various embodiments are foreseen to have valuable application as constituents of pharmaceutical preparations to treat various conditions generally defined as pathologies. Accordingly, embodiments may also comprise pharmaceutical compositions comprising nanoparticles in association with a pharmaceutically acceptable carrier. Preferably these compositions are in unit dosage forms such as tablets, pills, capsules, powders, granules, sterile parenteral solutions or suspensions, metered aerosol or liquid sprays, drops, ampoules, auto-injector devices or suppositories; for oral, parenteral, intranasal, sublingual or rectal administration, or for administration by inhalation or insufflation.

For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of nanoparticles described herein. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the nanoparticles described herein. Typical unit dosage forms contain from 1 to 100 mg, for example 1, 2, 5, 10, 25, 50 or 100 mg, of the active ingredient. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, acetyl alcohol and cellulose acetate. The compositions may be contained in a vial, sponge, syringe, tube, or other suitable container.

Liquid form preparations include solutions, suspensions, and emulsions, for example, water or water/propylene glycol solutions. For parenteral injection, liquid preparations can be formulated in solution in aqueous polyethylene glycol solution.

The pharmaceutical compositions may additionally include components to provide sustained release and/or comfort. Such components include high molecular weight, anionic mucomimetic polymers, gelling polysaccharides, and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes.

The pharmaceutical composition may be intended for intravenous use. The pharmaceutically acceptable excipient can include buffers to adjust the pH to a desirable range for intravenous use. Many buffers including salts of inorganic acids such as phosphate, borate, and sulfate are known.

Dosage

The dose administered to an animal, particularly a human, in accordance with the present disclosure should be sufficient to affect the desired response in the animal over a reasonable time frame. One skilled in the art will recognize that dosage will depend upon a variety of factors, including the strength of the particular compositions employed, the age, species, condition, and body weight of the animal. The size of the dose also will be determined by the route, timing and frequency of administration as well as the existence, nature, and extent of any adverse side effects that might accompany the administration of a particular composition and the desired physiological effect. It will be appreciated by one of ordinary skill in the art that various conditions or desired results, may require prolonged treatment involving multiple administrations.

Suitable doses and dosage regimens can be determined by conventional range-finding techniques known to those of ordinary skill in the art. Generally, treatment is initiated with smaller dosages, which are less than the optimum dose of the compound. Thereafter, the dosage is increased by small increments until the optimum effect under the circumstances is reached.

As used herein, by the term “effective amount,” “amount effective,” “therapeutically effective amount,” or the like, it is meant an amount effective at dosages and for periods of time necessary to achieve the desired result. The amount of the nanoparticles or nanoparticle containing composition administered per dose or the total amount administered per day may be predetermined or it may be determined on an individual patient basis by taking into consideration numerous factors, including the nature and severity of the patient's condition, the condition being treated, the age, weight, and general health of the patient, the tolerance of the patient to the compound, the route of administration, pharmacological considerations such as the activity, efficacy, pharmacokinetics and toxicology profiles of the compound and any secondary agents being administered, and the like. Patients undergoing such treatment will typically be monitored on a routine basis to determine the effectiveness of therapy. Continuous monitoring by the physician will insure that the optimal amount of the nanoparticles will be administered at any given time, as well as facilitating the determination of the duration of treatment. This is of particular value when secondary agents are also being administered, as their selection, dosage, and duration of therapy may also require adjustment. In this way, the treatment regimen and dosing schedule can be adjusted over the course of therapy so that the lowest amount of compound that exhibits the desired effectiveness is administered and, further, that administration is continued only so long as is necessary to successfully achieve the optimum effect.

Toxicity

The ratio between toxicity and therapeutic effect for a particular compound (nanoparticle) is its therapeutic index and can be expressed as the ratio between LD₅₀ (the amount of compound lethal in 50% of the population) and ED₅₀ (the amount of compound effective in 50% of the population). Therapeutic agents that exhibit high therapeutic indices are preferred. Therapeutic index data obtained from cell culture assays and/or animal studies can be used in formulating a range of dosages for use in humans. The dosage of such compounds preferably lies within a range of plasma concentrations that include the ED₅₀ with little or no toxicity. The dosage may vary within this range depending upon the dosage form employed and the route of administration utilized. See, e.g. Fingl et al., In: THE PHARMACOLOGICAL BASIS OF THERAPEUTICS, Ch. 1, p. 1, 1975. The exact formulation, route of administration, and dosage can be chosen by the individual physician in view of the patient's condition and the particular method in which the therapeutic agent is used.

When parenteral application is needed or desired, particularly suitable admixtures for the nanoparticles having a therapeutic agent included in the pharmaceutical composition may be injectable, sterile solutions, oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-block polymers, and the like. Ampoules are convenient unit dosages. Pharmaceutical admixtures suitable for use in the pharmaceutical compositions presented herein may include those described, for example, in Pharmaceutical Sciences (17th Ed., Mack Pub. Co., Easton, Pa.) and WO 96/05309, the teachings of both of which are hereby incorporated by reference.

Certain embodiments of the nanoparticles provided herein may be supplied as kits. The kits may further include instruction for use. The kit may supply the nanoparticles provided herein as separate components that may be assembled into nanoparticles using included instructions.

The following examples are provided as an aid in examining particular aspects of the invention, and represent only certain embodiments and explanations of embodiments. The examples are in no way meant to be limiting of the invention scope. The materials and methods provided below are those which were used in performing the examples that follow.

### EXAMPLES

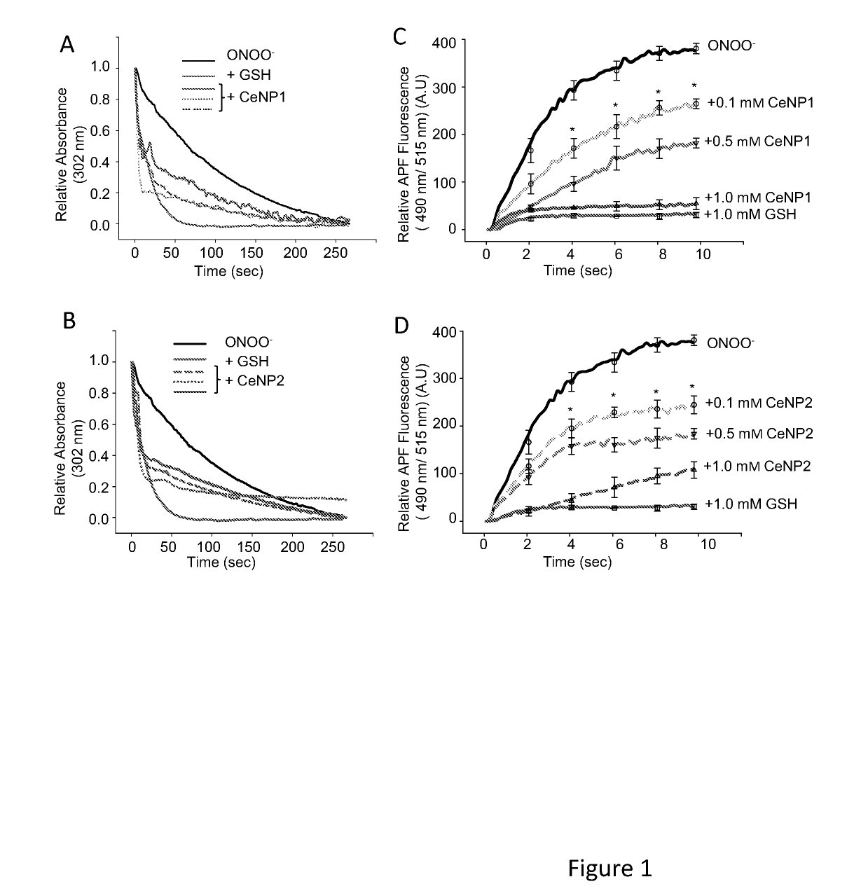

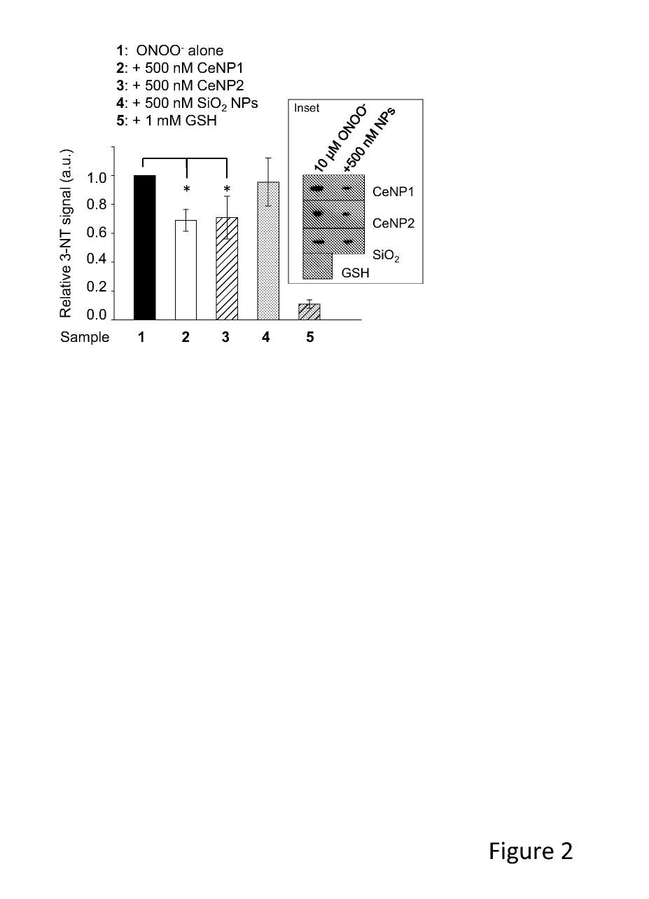

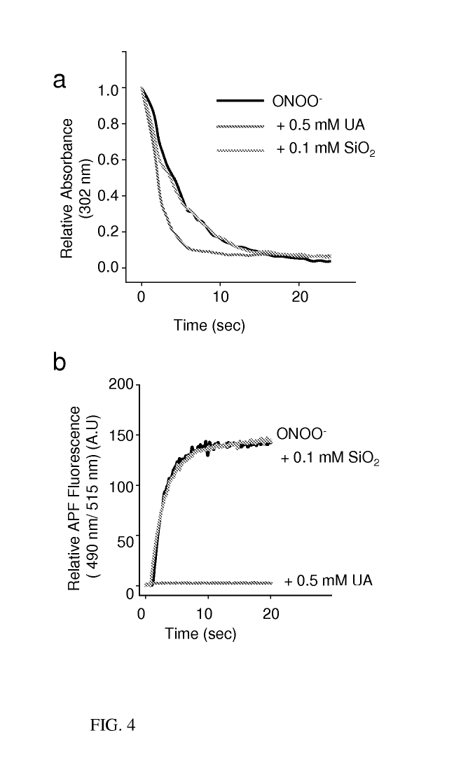

Peroxynitrite absorbs UV light at a wavelength of 302 nm, thus its decay can be measured kinetically using UV-spectroscopy [12]. Exploiting this property, it was observed that two water-based preparations of CeO₂ NPs dramatically accelerated the normal rapid decay of peroxynitrite (FIG. 1). CeNP1 (with increased Ce³⁺ in their outer surface) (FIG. 1a) or CeNP2 (with decreased Ce³⁺ in their outer surface) (FIG. 2b) both accelerated the decay in manner similar to an established antioxidant, glutathione (GSH). The recognized peroxynitrite scavenger uric acid [13] was also tested and similar results were found (FIG. 4a). When an unrelated metal oxide NP of similar size, SiO₂, was tested, (FIG. 4a) there was no change in the rate of ONOO⁻ decay suggesting this was somewhat unique to CeO₂ NPs. A second addition of peroxynitrite after decay again was accelerated in the presence of cerium oxide nanoparticles, suggesting strongly that these materials were acting as catalysts and not just reacting with peroxynitrite to generate a modified cerium oxide material (data not shown).

To test the reactivity of CeO₂ NPs with peroxynitrite by an alternative method, the oxidation of 3′-(p-aminophenyl) fluorescein (APF) by fluorescence spectrometry was followed in vitro. APF has no fluorescence at baseline, but when oxidized by peroxynitrite, it exhibits fluorescence [14]. Using this probe, it was observed that CeNP1 and CeNP2 prevented the oxidation of APF in vitro, similar to GSH when challenged with peroxynitrite (FIGS. 1c and d) [15]. Again, the same controls were tested in this assay with similar results as before with uric acid and SiO₂ NPs (FIG. 4b). Collectively, these data illustrate a previously unknown catalytic property of CeO₂ NPs, namely their ability to accelerate the decay of peroxynitrite in vitro.

Protection against peroxynitrite-induced damage in vivo is provided by several antioxidants [16] as well as eliminated by many of the antioxidant enzymes including peroxiredoxins [17]. Modification of amino acid residues on the active site of key proteins may result in detrimental change in function of the proteins. Tyrosine nitration modification of proteins has become an important biomarker for inflammation and for nitrosative stress and has been detected in a number of diseases and pathological conditions [11]. Tyrosine nitration occurs with the incorporation of a nitro group (—NO₂) at position 3 of the aromatic ring of tyrosine [18].

To demonstrate the biological significance of CeO₂ NPs with ONOO⁻, it was tested whether CeO₂ NPs would reduce the level of peroxynitrite-induced protein tyrosine nitration of the protein bovine serum albumin (BSA) using a specific antibody for 3-nitrotyrosine. In FIG. 2, using quantitative densitometry analyses, it was observed that bovine serum albumin (BSA), treated with 10 μM peroxynitrite, exhibited high 3-nitrotyrosine immunoreactivity (FIG. 2, lane 1) as expected due to the production of nitrated tyrosines on the surface. By contrast, the same experiment carried out in the presence CeNP1, CeNP2 or GSH showed a significant decrease in tyrosine nitration (FIG. 2, lanes 2, 3 & 5 and inset). Addition of SiO₂ NPs had no effect (FIG. 2, lane 4 and inset). These results further demonstrate that CeO₂ NPs can interact with peroxynitrite or its breakdown products thus preventing the 3-NT modification of BSA in a manner similar to glutathione.

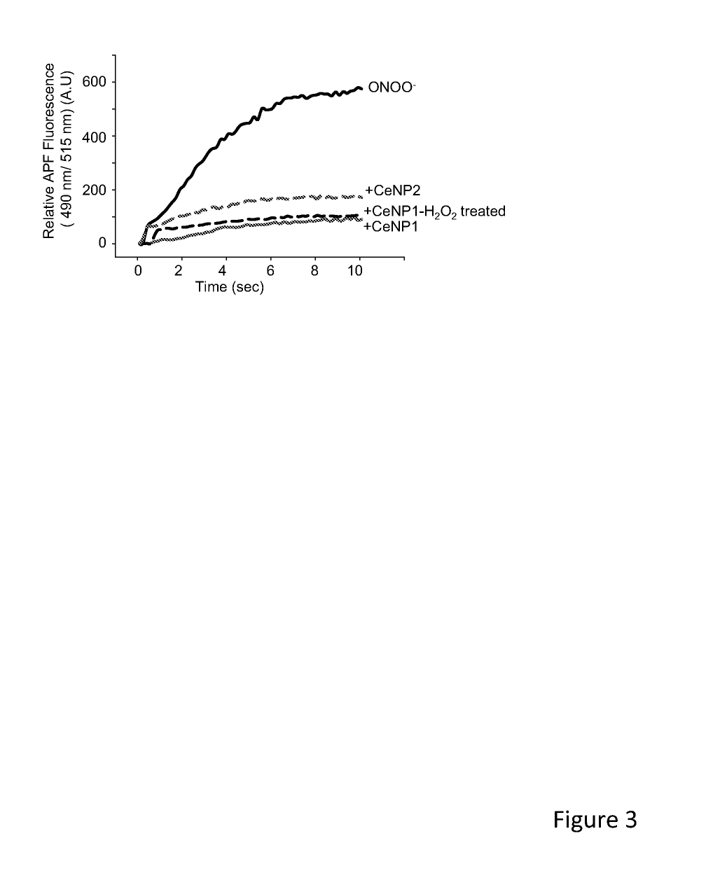

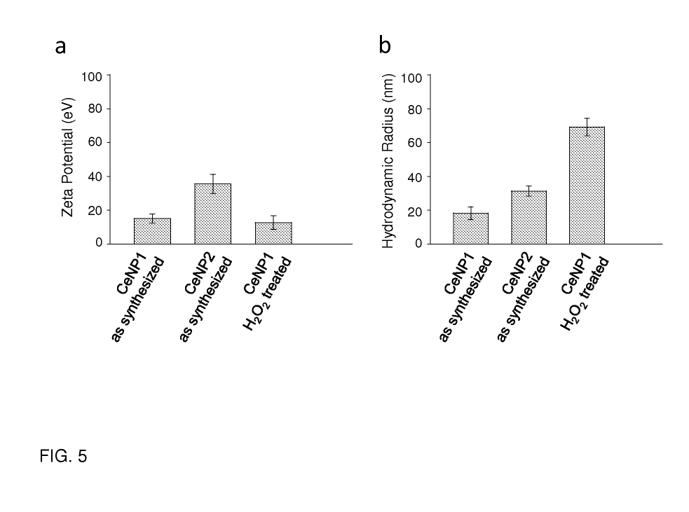

CeO₂ NPs have a mixed valence state of cerium containing both Ce³⁺ and Ce⁴⁺. It has been shown that upon incubation of CeO₂ NPs with hydrogen peroxide, CeO₂ NPs with a higher starting concentration of Ce³⁺ can convert to CeO₂ NPs containing increased Ce⁴⁺ on their surface [3]. Along with this change in oxidation state is the loss of their SOD mimetic ability. To determine whether this change in property also applies to ONOO⁻ interaction, CeNP1 was incubated with hydrogen peroxide and followed ONOO⁻ ability to oxidize APF (FIG. 3). Since CeNP2 had previously prevented the oxidation of APF (FIG. 1d), it was not surprising that H₂O₂ incubation did not affect CeNP1 ability to oxidize APF. Additionally, incubation with H₂O₂ did not affect zeta potential nor significantly affect their hydrodynamic radius (FIG. 5). These results suggest that the interaction with ONOO⁻ does not specifically depend upon the Ce³⁺ sites.

The biochemistry of ONOO⁻ is vastly complicated due to the multiple reactions possible in the presence and absence of CO₂, H⁺ and metals during its decomposition [18]. The acceleration of peroxynitrite decay observed in the presence of cerium oxide nanoparticles represents compelling yet preliminary evidence that these nanomaterials readily react with peroxynitrite or one of the reactive oxidants and radicals that are a result of the non-enzymatic breakdown of peroxynitrite. The mechanism by which these materials can alter the catalytic decomposition of peroxynitrite has yet to be elucidated. Others have shown that thiols, metals and carbon dioxide are the most likely targets of peroxynitrite in vivo [18, 19]. Not to be limited by theory, assuming cerium oxide reacts directly with peroxynitrite (and not decay products such as carbonate radical), a putative scheme of one electron oxidation and reduction reactions is shown that could explain the acceleration of peroxynitrite decay.

O2*⁻+.NO [symbol] ONOO⁻+H⁺→[.OH+.NO₂]^{[18]}

Ce³⁺+.NO₂→Ce⁴⁺+NO₂⁻

Ce³⁺+.OH→Ce⁴⁺+OH⁻

Ce³⁺+O2*⁻→Ce⁴⁺+O₂²⁻

2Ce⁴⁺+O₂²⁻→O₂+2Ce³⁺

The proton-dependent decay of peroxynitrite results in production of hydroxyl radical and nitrogen dioxide radical. Each of these potent radicals, which are one-electron oxidants, could react with cerium oxide nanoparticles at the particle surface (in an oxygen vacancy) to oxidize Ce³⁺ to Ce⁴⁺ with the concomitant release of hydroxyl ion or nitrite. In our UV-visible measurements an absorption peak near that of nitrite (229 nm) was observed that paralleled the decrease in peroxynitrite levels (data not shown).

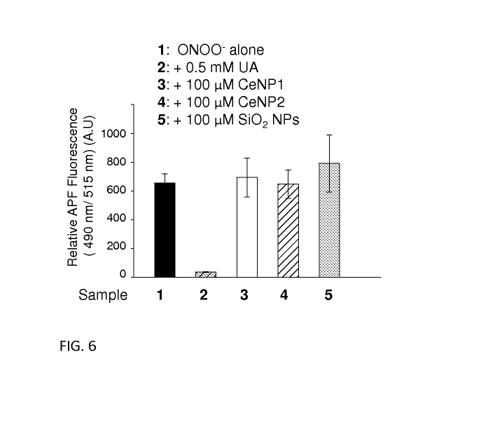

Additionally, CO₂ plays a dramatic role in the decomposition of ONOO⁻ [20]. In tissues, where concentrations of CO₂ can reach 1-2 mM, the reaction with peroxynitrite is highly favored forming nitrosoperoxycarboxylate anion adduct (ONOOCO₂⁻) that undergoes deleterious decay yielding carbonate radical (CO₃⁻) and nitrogen dioxide radical (.NO₂) [18]. To begin to understand whether the accelerated decay of ONOO⁻ in the presence of CeO₂ NPs was due to their interaction with the breakdown products of ONOO⁻, APF assays were repeated in an end-point format under inert conditions (argon atmosphere). Components were flushed with argon to remove as much normal atmosphere, including CO₂, as possible as well the assays were performed in an argon flushed hood. With the removal of CO₂, CeNP1 or CeNP2 were no longer able to accelerate the decay of ONOO⁻ (FIG. 6). This suggests that CeO₂ NPs could be interacting with carbonate radical known to be formed during the decomposition of ONOO⁻. It is well established that in air saturated buffer the carbonate radical is the most reactive radical species present during the spontaneous decay of peroxynitrite, suggesting it is the likely target of reaction with CeO₂ NPs.

It has been suggested in many studies that protection that has been observed in cell culture and animal studies is due to the presence of CeO₂ NPs and their ability to react with or scavenge the major deleterious ROS/RNS species [21-23]. Globally, it appears that there are going to be different mechanisms for CeO₂ NPs with varying ratio of Ce³⁺/Ce⁴⁺ at the particle surface and probable different mechanisms for the various species of ROS/RNS the NPs may encounter. In an attempt to correlate catalytic activity to surface oxidation, it has been found that CeO₂ NPs that have higher levels of reduced cerium sites (3+) at the surface are more effective SOD mimetics [3, 5]. In contrast, CeO₂ NPs that have fewer reduced cerium sites exhibit better catalase mimetic [6] and .NO scavenging capabilities [7]. The results from this study suggest that in the presence of CeO₂ NPs, regardless of the oxidation state, peroxynitrite, or one of its breakdown products may react with the surface of CeO₂ NPs. The precise molecular mechanism behind each of these catalytic reactions is still not yet known, but research on this issue is ongoing. What happens in the milieu of cells and tissues remains an enigma.

### Materials and Methods Related to Examples

Cerium nitrate hexahydrate (99.999% pure from Sigma Aldrich, St. Louis, Mo.) were used as a precursor for all of the preparations. Anti-3-nitrotyrosine (3-NT) and hydrogen peroxide were also purchased from Sigma Aldrich. Glutathione (GSH) and diethylenetriaminepentaacetic acid (DTPA) were purchased from Fisher Scientific (Pittsburgh, Pa.). Bovine serum albumin (BSA) was purchased from Pierce Biotechnology, Inc., (Rockford, Ill.). SiO₂ nanoparticles were purchased from Corpuscular Inc. (Cold Spring, N.Y.). Peroxynitrite and 2-[6-(4-aminophenoxy)-3-oxo-3H-xanthen-9-yl]-benzoic acid (APF) were purchased from Cayman Chemicals (Ann Arbor, Mich.).

Nanoparticle Synthesis and Characterization

Cerium oxide nanoparticles with a higher Ce³⁺/Ce⁴⁺ ratio (CeNP1) or with lower Ce³⁺/Ce⁴⁺ ratio (CeNP 2) were prepared using wet chemical method as described previously (Das, S., Singh, S., Dowding, J. M., Oommen, S., Kumar, A., Sayle, T. X., Saraf, S., Patra, C. R., Vlahakis, N. E., Sayle, D. C., Self, W. T., and Seal, S. (2012) The induction of angiogenesis by cerium oxide nanoparticles through the modulation of oxygen in intracellular environments, Biomaterials.; Patil, S., Kuiry, S. C., Seal, S., and Vanfleet, R. (2002) Synthesis of nanocrystalline ceria particles for high temperature oxidation resistant coating, J Nanopart Res 4, 433-438). In brief, to prepare CeNP1 with a high ratio of Ce³⁺/Ce⁴⁺, Ce (NO₃)₃ .6H₂O (5 mM) was dissolved in sterile dH₂O. While stirring the nitrate precursor, H₂O₂ (2% v/v) was rapidly added while stirring at 300 rpm for 15 min. The solution was continuously stirred for additional 1 h to obtain a stable dispersion of cerium oxide nanoparticles. CeNP2 with a low ratio of Ce³⁺/Ce⁴⁺ was synthesized using ammonium hydroxide (NH₄OH) precipitation method. Briefly, cerium nitrate hexahydrate was dissolved in sterile dH₂O and stoichiometric amount of NH₄OH was added and stirred for an additional 4 h at room temperature. Cerium oxide nanoparticles were collected by centrifugation at 8000 g for 10 min. Samples were stored at room temperature. All preparations were sonicated to ensure single nanoparticles (Branson, Danbury, Conn.) prior to use. Hydrodynamic radius and surface charge (zeta potential) of the nanoparticles were estimated using Zetasizer (Nano-ZS from Malvern Instruments, Houston, Tex.). The surface chemistry of the cerium oxide nanoparticles confirming Ce³⁺/Ce⁴⁺ ratio was determined by X-ray photoelectron spectroscopy (XPS) and has been previously reported (Dowding, J. M., Dosani, T., Kumar, A., Seal, S., and Self, W. T. (2012) Cerium oxide nanoparticles scavenge nitric oxide radical (NO), Chem Commun (Camb) 48, 4896-4898).

Peroxynitrite Decay Using Spectroscopy.

Peroxynitrite (20 μM) was added while stirring into a 1 ml quartz cuvette with a 1 cm path length. Each sample was analyzed for a total of 600 seconds with a cycle time of 0.5 seconds at a wavelength of 302 nm in 100 mM sodium or potassium phosphate buffers, pH 9.5, and 100 μM diethylenetriaminepentaacetic acid (DPTA) to minimize any potential interference by adventitious metal ions using a Hewlett-Packard diode array UV-visible 8453 spectrophotometer. Absorbance was normalized by subtracting the final absorbance from initial absorbance and dividing by the amplitude as previously described (Quijano, C., Hernandez-Saavedra, D., Castro, L., McCord, J. M., Freeman, B. A., and Radi, R. (2001) Reaction of Peroxynitrite with Mn-Superoxide Dismutase: ROLE OF THE METAL CENTER IN DECOMPOSITION KINETICS AND NITRATION, Journal of Biological Chemistry 276, 11631-11638.).

Peroxynitrite Decay Using APF In Vitro.

APF (10 μM) fluorescence was measured at excitation/emission wavelengths of 490 nm/515 nm in 100 mM nitrogen flushed sodium phosphate buffer, pH 7.4, containing 100 μM DPTA using a Varian Cary Eclipse fluorescence spectrophotometer (Palo Alto, Calif.). Fluorescence was followed for 1 min at room temperature using a quartz fluorometer cell (Starna Cells, Inc. Atascadero, Calif.). Peroxynitrite was added last due to the short-half of peroxynitrite at pH 7.4. As stock solutions of ONOO⁻ contain 0.3 M NaOH, control incubations were performed with equivalent amounts of NaOH.

Slot Blot Assay for 3-Nitrotyrosine.

500 ng bovine serum albumin (BSA) was treated with 10 μM ONOO⁻ in the absence and presence of 500 nM NPs or 1 mM GSH at room temperature for 20 minutes. Nitrocellulose (Hybond ECL, GE Healthcare) was equilibrated using 1× tris buffered saline (TBS) and placed in Mini-fold Slot-Blot System (GE Healthcare). Reactions were pipetted onto the membrane and allowed to incubate for 20 min. After 3 washes, the membrane was blocked in 5% milk, 1×TBS+0.05% Tween. The blot was probed with antibody specific for 3-nitro-tyrosine modification at a 1:2000 dilution (Sigma) followed by horseradish peroxidase-conjugated secondary at a 1:15,000 dilution (ECL). For detection, West Dura substrate (Thermo Scientific) was used as per manufacturer's suggested protocol. Densitometry analysis (ImageJ Software) was carried out to quantify nitrotyrosine levels. Statistical significance of varying number of experiments was determined using a two-tailed non-paired Student's t-test and were calculated by using SigmaPlot® 10 software (Systat Software, Inc., Point Richmond, Calif.). Actual slot blot experiments performed was as follows: peroxynitrite alone, four replicates; CeNP1, four replicates; CeNP2, three replicates; SiO₂, three replicates; GSH, four replicates.

Hydrogen Peroxide Pretreatment of CeNP1.

CeNP1s were treated with hydrogen peroxide (100 mM) for 24 h. The color of the nanoparticles changed from clear to yellow indicating a shift in oxidation state from +3 to +4 as previously described (Heckert, E. G., Karakoti, A. S., Seal, S., and Self, W. T. (2008) The role of cerium redox state in the SOD mimetic activity of nanoceria, Biomaterials 29, 2705-2709). Actual traces performed was as follows: peroxynitrite alone, six replicates; CeNP2, three replicates; CeNP1-H₂O₂ treated, three replicates; CeNP1, three replicates.

### REFERENCES

1. Harman D. Aging and disease: Extending functional life span. Ann N Y Acad Sci. 1996; 786:321-36.
2. Drew B, Leeuwenburgh C. Aging and the role of reactive nitrogen species. Ann N Y Acad Sci. 2002; 959:66-81.
3. Heckert E G, Karakoti A S, Seal S, Self W T. The role of cerium redox state in the sod mimetic activity of nanoceria. Biomaterials. 2008; 29:2705-9.
4. Sun C W, Li H, Chen L Q. Nanostructured ceria-based materials: Synthesis, properties, and applications. Energy & Environmental Science. 2012; 5:8475-505.
5. Korsvik C, Patil S, Seal S, Self W T. Superoxide dismutase mimetic properties exhibited by vacancy engineered ceria nanoparticles. Chem Commun (Camb). 2007:1056-8.
6. Pirmohamed T, Dowding J M, Singh S, Wasserman B, Heckert E, Karakoti A S, et al. Nanoceria exhibit redox state-dependent catalase mimetic activity. Chem Commun (Camb). 2010; 46:2736-8.
7. Dowding J M, Dosani T, Kumar A, Seal S, Self W T. Cerium oxide nanoparticles scavenge nitric oxide radical (no). Chem Commun (Camb). 2012; 48:4896-8.
8. Batinić-Haberle I. Manganese porphyrins and related compounds as mimics of superoxide dismutase. In: Lester P, editor. Methods in enzymology: Academic Press; 2002. p. 223-33.
9. Day B J, Fridovich I, Crapo J D. Manganic porphyrins possess catalase activity and protect endothelial cells against hydrogen peroxide-mediated injury. Archives of Biochemistry and Biophysics. 1997; 347:256-62.
10. Ferrer-Sueta G, Vitturi D, Batinic-Haberle I, Fridovich I, Goldstein S, Czapski G, et al. Reactions of manganese porphyrins with peroxynitrite and carbonate radical anion. J Biol Chem. 2003; 278:27432-8.
11. Pacher P, Beckman J S, Liaudet L. Nitric oxide and peroxynitrite in health and disease. Physiol Rev. 2007; 87:315-424.
12. Radi R. Kinetic analysis of reactivity of peroxynitrite with biomolecules. Methods Enzymol. 1996; 269:354-66.
13. Pompella A, Visvikis A, Paolicchi A, De Tata V, Casini A F. The changing faces of glutathione, a cellular protagonist. Biochem Pharmacol. 2003; 66:1499-503.
14. Setsukinai K, Urano Y, Kakinuma K, Majima H J, Nagano T. Development of novel fluorescence probes that can reliably detect reactive oxygen species and distinguish specific species. J Biol Chem. 2003; 278:3170-5.
15. Whiteman M, Halliwell B. Protection against peroxynitrite-dependent tyrosine nitration and alpha 1-antiproteinase inactivation by ascorbic acid. A comparison with other biological antioxidants. Free Radic Res. 1996; 25:275-83.
16. Balavoine G G, Geletii Y V. Peroxynitrite scavenging by different antioxidants. Part i: Convenient assay. Nitric Oxide. 1999; 3:40-54.
17. Poole L B, Hall A, Nelson K J. Overview of peroxiredoxins in oxidant defense and redox regulation. Curr Protoc Toxicol. 2011; Chapter 7:Unit7 9.
18. Pietraforte D, Salzano A M, Marino G, Minetti M. Peroxynitrite-dependent modifications of tyrosine residues in hemoglobin. Formation of tyrosyl radical(s) and 3-nitrotyrosine. Amino Acids. 2003; 25:341-50.
19. Beckman J S. Oxidative damage and tyrosine nitration from peroxynitrite. Chem Res Toxicol. 1996; 9:836-44.
20. Pryor W A, Lemercier J N, Zhang H, Uppu R M, Squadrito G L. The catalytic role of carbon dioxide in the decomposition of peroxynitrite. Free Radic Biol Med. 1997; 23:331-8.
21. Chen J, Patil S, Seal S, McGinnis J F. Rare earth nanoparticles prevent retinal degeneration induced by intracellular peroxides. Nat Nano. 2006; 1:142-50.
22. Estevez A Y, Pritchard S, Harper K, Aston J W, Lynch A, Lucky J J, et al. Neuroprotective mechanisms of cerium oxide nanoparticles in a mouse hippocampal brain slice model of ischemia. Free Radic Biol Med. 2011; 51:1155-63.
23. Kim C K, Kim T, Choi I Y, Soh M, Kim D, Kim Y J, et al. Ceria nanoparticles that can protect against ischemic stroke. Angew Chem Int Ed Engl. 2012.

It should be borne in mind that all patents, patent applications, patent publications, technical publications, scientific publications, and other references referenced herein are hereby incorporated by reference in this application in order to more fully describe the state of the art to which the present disclosure pertains.

Reference to particular buffers, media, reagents, cells, culture conditions and the like, or to some subclass of same, is not intended to be limiting, but should be read to include all such related materials that one of ordinary skill in the art would recognize as being of interest or value in the particular context in which that discussion is presented. For example, it is often possible to substitute one buffer system or culture medium for another, such that a different but known way is used to achieve the same goals as those to which the use of a suggested method, material or composition is directed.

It is important to an understanding of the present invention to note that all technical and scientific terms used herein, unless defined herein, are intended to have the same meaning as commonly understood by one of ordinary skill in the art. The techniques employed herein are also those that are known to one of ordinary skill in the art, unless stated otherwise. For purposes of more clearly facilitating an understanding the invention as disclosed and claimed herein, the following definitions are provided.

While a number of embodiments of the present disclosure have been shown and described herein in the present context, such embodiments are provided by way of example only, and not of limitation. Numerous variations, changes and substitutions will occur to those of skill in the art without materially departing from the invention herein. For example, the present invention need not be limited to best mode disclosed herein, since other applications can equally benefit from the teachings of the present invention. Also, in the claims, means-plus-function and step-plus-function clauses are intended to cover the structures and acts, respectively, described herein as performing the recited function and not only structural equivalents or act equivalents, but also equivalent structures or equivalent acts, respectively. Accordingly, all such modifications are intended to be included within the scope of this invention as defined in the following claims, in accordance with relevant law as to their interpretation.

What is claimed is:

1. A method of reducing the adverse effects of peroxynitrite in a subject, said method comprising administering a therapeutically effective amount of cerium oxide nanoparticles to said subject.

2. The method of claim 1, wherein said cerium oxide nanoparticles accelerate decay of said peroxynitrite independent of the valence state of cerium oxide nanoparticles.

3. The method of claim 1, wherein said cerium oxide nanoparticles have a higher 3+/4+ ratio, lower 3+/4+ ratio, or a ratio therebetween.

4. A method of treating a subject with elevated levels of peroxynitrite, comprising accelerating decay of the peroxynitrite via administering a therapeutically effective amount of cerium oxide nanoparticles to the subject, wherein the cerium oxide nanoparticles reduce the level of peroxynitrite in the subject.

5. The method of claim 4, wherein the cerium oxide nanoparticles range between 1-5 nanometers in size.

6. The method of claim 4, wherein the cerium oxide nanoparticles range between 5-10 nanometers in size.

7. The method of claim 4, wherein said cerium oxide nanoparticles scavenge peroxynitrite or breakdown products thereof.

8. A method of treating a subject identified as at risk of developing or which has a neurodegenerative disease, comprising:
administering a therapeutically effective amount of cerium oxide nanoparticles to the subject, wherein the cerium oxide nanoparticles scavenge reactive peroxynitrite in the subject independent of valence state.

9. The method of claim 8, wherein the subject exhibits symptoms comprising at least one of the following: amyloid plaques in the brain or increased beta-amyloid deposits, neurofibrillary tangles (NFTs) in the brain, an atrophic brain region, atrophy of the hippocampus, increase in brain ventricle size, elevated levels of tau and/or amyloid beta proteins in cerebral spinal fluid, loss of function in the temporal lobe, cognitive deficiencies in memory, language, perceptual skills, attention, constructive abilities, orientation, problem solving, and/or functional abilities.

10. The method of claim 8, wherein the subject exhibits symptoms comprising at least two of the following: amyloid plaques in the brain or increased beta-amyloid deposits, neurofibrillary tangles (NFTs) in the brain, an atrophic brain region, atrophy of the hippocampus, increase in brain ventricle size, elevated levels of tau and/or amyloid beta proteins in cerebral spinal fluid, loss of function in the temporal lobe, cognitive deficiencies in memory, language, perceptual skills, attention, constructive abilities, orientation, problem solving, and/or functional abilities; whereby said at least two symptoms are treated by said administering of said nanoparticles.

11. The method of claim 8, wherein the subject exhibits symptoms comprising at least one of the following: reduced dopaminergic activity in the basal ganglia, death of dopaminergic neurons in the substantia nigra pars compacta, motor symptoms comprising Parkinsonian gait, tremors, rigidity, slowness of movement, postural instability, non-motor symptoms comprising autonomic dysfunction, neuropsychiatric problems, sensory problems and/or sleep-related difficulties.

12. The method of claim 8, wherein the subject exhibits symptoms comprising at least two of the following: reduced dopaminergic activity in the basal ganglia, death of dopaminergic neurons in the substantia nigra pars compacta, motor symptoms comprising Parkinsonian gait, tremors, rigidity, slowness of movement, postural instability, non-motor symptoms comprising autonomic dysfunction, neuropsychiatric problems, sensory problems and/or sleep-related difficulties; whereby said at least two symptoms are treated by said administering of said nanoparticles.

13. A method of reducing brain inflammation in a patient, comprising:
administering a therapeutically effective amount of cerium oxide nanoparticles to the patient, said cerium oxide nanoparticles effective to reduce levels of peroxynitrite, or breakdown products thereof, in the brain.

14. A method of scavenging reactive nitrogen species in a subject, comprising:
administering a therapeutically effective amount of cerium oxide nanoparticles to the subject, wherein said cerium oxide nanoparticles associate with a membrane in the subject and accelerate decomposition of the reactive nitrogen species in the subject, wherein the reactive nitrogen species is peroxynitrite or breakdown products thereof.

15. The method of claim 14, wherein the membrane is a mitochondrial membrane and/or a plasma membrane.

16. The method of claim 1, further comprising testing for a change in peroxynitrite levels or symptoms associated with elevated peroxynitrite levels, or symptoms of disease associated with elevated peroxynitrite levels following said administering.

17. The method of claim 16, further comprising changing a dosage level or dosage frequency following said testing.

18. The method of claim 10, wherein treating said at least two symptoms comprises reducing the level of said symptoms or reducing the progression of said at least two symptoms over a predetermined period of time.

19. The method of claim 12, wherein treating said at least two symptoms comprises reducing the level of said symptoms or reducing the progression of said at least two symptoms over a predetermined period of time.

20. The method of claim 14, further comprising testing for a change in peroxynitrite levels or symptoms associated with elevated peroxynitrite levels, or symptoms of disease associated with elevated peroxynitrite levels following said administering.
